(19) United States
(12) Patent Application Publication
(10) Pub. No.: US 2022/0085617 A1
(43) Pub. Date: Mar. 17, 2022

(54) USE OF A MOBILE ENERGY CONVERSION SYSTEM FOR OPERATING A MEDICAL DEVICE AND A STERILE GOODS CYCLE MONITORING SYSTEM

(71) Applicant: Aesculap AG, Tuttlingen (DE)

(72) Inventors: Andreas Elisch, Dunningen (DE); Matthias Henke, Villingen-Schwenningen (DE)

(21) Appl. No.: 17/476,535

(22) Filed: Sep. 16, 2021

(30) Foreign Application Priority Data
Sep. 17, 2020 (DE) 10 2020 124 319.7

(51) Int. Cl. H02J 7/00; H02J 3/32; H02J 7/34; H02N 99/00; A61B 50/13

(52) U.S. Cl. CPC H02J 7/0013; H02J 3/32; A61B 50/13; H02J 7/345; H02N 99/00; H02J 7/0044

(57) ABSTRACT

An energy conversion system for converting kinetic energy into electrical energy and for supplying electricity to at least one electric component of a medical device, which may be a transport and/or storage device for sterile goods, such as a cabinet carriage, a shelf carriage, a supply table or a removal carriage. The electrical energy is generated from movement of at least one part of the medical device and/or the entire medical device. The energy conversion system can be used to convert kinetic energy into electrical energy for making a medical device available. The medical device can include at least one electric component and a monitoring system a sterile goods cycle.

### CROSS-REFERENCE TO RELATED APPLICATION

This application claims the benefit of priority of German Application No. DE 10 2020 124 319.7, filed Sep. 17, 2020, the content of which is incorporated by reference herein in its entirety.

### FIELD

The present disclosure relates to the use of a (modular) mobile energy conversion system for supplying at least one electric component of a medical, preferably movable/mobile, device, to the use of the (modular) mobile energy conversion system for providing the medical, preferably mobile, device including at least one electric component, and to a monitoring system of a sterile goods cycle equipped with the medical device including the (modular) mobile energy conversion system, in particular for tracking and data exchange.

### BACKGROUND

In a hospital, it is basically very important to know where and in which process step particular instruments, in particular sterile goods, are located, as no operation can be carried out, for example, without the correct sterile goods (in the proper amount and the proper quality). Therefore, primarily the sterile goods treatment requires utmost hygiene, precision and reliability. The sterile goods treatment is thus part of the most important and, at the same time, most sensitive fields of a hospital. Here it is always a question of promptness and safety.

All instruments must pass a specific sequence of working steps and, additionally, must be inventoried and stored in the case of non-use. For the purpose of sterilization, transport and storage, the sterile goods are packaged in soft packages or sterile goods containers. The soft packages are partially accommodated in additional boxes. The storage itself takes place in open or closed shelf systems in specifically dedicated rooms. The transport between sterilization and warehouse or between warehouse and operating theater and, resp., from the operating theater to the central sterile goods treatment department (hereinafter, the “ZSVA”) is usually carried out in appropriate carriages. Depending on the requirement, use is made of movable cabinet carriages, shelf carriages or movable supply tables or removal tables. This requires high density of documentation and serves for preventing complaints as well as for the patient safety. A complete documentation system provides proof of which employee was entrusted with the processing in which process step and which processes were used.

The sterile goods treatment must ensure that, during use, the treated medical product entails no risk of damage to health in accordance with infections, pyrogenic, allergic and toxic reactions. Therefore, for risk minimization, it is of advantage that the instruments are provided with a label, preferably an RFID tag, and can be read out in combination with the containers/soft packages via antennas, and, in this way, the working steps passed can be automatically detected and documented. Currently, sterilized instruments are stored in containers and are provided with a seal as a label of sterility. Accordingly, the sterile goods cannot be used any more, if a seal is damaged, irrespective of whether or not the instruments would still meet the requirements of sterility.

For such automatic detection process, the power supply is essential for reading out and/or, where appropriate, writing on the installed tags and antennas for data transmission systems, the storage and communication units etc.

In FIG. 3, a generally known sterile goods cycle is summarized to the essential steps or stations. As a result, in particular in the fields of “supply”, “use” as well as “checking, care, functional test”, “packaging, labeling” medical products are excessively moved and, therefore, temporarily fall out of the installed monitoring system. In other words, at least these fields are monitoring voids so that continuous close documentation of the location of sterile goods is not possible.

Furthermore, it has shown that sterile goods are usually located, in particular in the afore-mentioned monitoring voids, in/on movable/mobile transport/storage devices, particularly the afore-mentioned cabinet carriages, shelf carriages or else movable supply tables or removal tables. Although it is imaginable to equip these movable/mobile transport/storage devices equally with monitoring electronics to obtain an as complete documentation of the sterile goods cycle as possible, these monitoring electronics must also be supplied with energy, however, that can be taken, for example, from the local mains and/or from an intermediate energy accumulator (battery), where appropriate.

However, the problem in this context is that the reliability of the energy supply depends on the diligence of the operator who must ensure, either always or at least at regular intervals, a power supply from the local mains. For solving this problem, e.g., alarm systems would be imaginable which in time indicate an imminent energy undersupply. However, those warning systems are not only annoying especially in the daily medical routine, but may also be irritating, as usually they are indicative of a dangerous situation, which would not be the case here, and therefore have to be used sparingly.

### SUMMARY

Against this background, it is an object of the present disclosure to provide a possibility of reliably supplying sufficient energy to an electrical component of a medical movable/mobile device, in particular a movable/mobile transport/storage device for sterile goods, and in this way largely prevent an energy undersupply.

This object is achieved by the use of an energy conversion system converting kinetic energy into electrical energy on manually movable parts such as wheels, drawers, doors, etc. of the medical movable/mobile device.

Consequently, the core of the present disclosure consists in initially detecting the frequency of the occurrence of a movement/actuation of the respective movable parts of the medical movable/mobile device concerned within the scope of a sterile goods cycle and in evaluating therefrom the kinetic total energy. Depending on this evaluation, it is then possible to arrange generators e.g. in the form of dynamos or any other energy converting equipment of this species to specific parts rich in kinetic energy of the medical movable/mobile device and, thus, to harvest the kinetic energy of the respective parts in the form of electrical energy.

It has turned out according to the disclosure that other forms of energy such as heat or light are not sufficiently yielding in order to guarantee the self-sustaining energy supply of an electric component of a medical movable/mobile device, in particular of a movable/mobile transport/storage device for sterile goods in the long run, and therefore the installation thereof is uneconomical. However, the inventor found that, particularly within the scope of a generally known sterile goods cycle, movable parts of the medical movable/mobile device, in particular transport/storage devices, preferably cabinet carriages, shelf carriages or movable supply tables or removal tables as well as the medical movable/mobile device itself are moved/actuated so frequently that the kinetic energy convertible therefrom is sufficient to charge, for example, an electrical energy accumulator for supplying the electric component and, thus, to safeguard continuous power supply.

In other words, the afore-mentioned object is achieved in the present disclosure by the use of such a modular energy conversion system for supplying at least one electric component of a medical movable device, preferably a cabinet carriage, shelf carriage, supply table and/or removal carriage, which is provided and configured as well as arranged/placed to convert a movement of at least one part of the movable device and/or of the whole movable device itself and, resp., the kinetic energy generated in this way into electrical energy.

Accordingly, the modular energy conversion system is preferably configured with a generator, a controller and an energy storage unit. The generator is arranged and/or mounted on at least one part, preferably on at least one roller/wheel of the movable medical device (transport/storage device for sterile goods), wherein the part was selected under the aspect of the frequency of its (manual) actuation within the scope of a sterile goods cycle. The controller, preferably a charge controller, is provided and configured to convert the electrical energy into DC voltage, and the energy accumulator/energy storage unit is provided and configured to intermediately store the DC voltage output by the controller and to transmit the DC voltage to a consumer in response to at least one trigger point.

In other words, by the movement of individual components or the whole movable device, the resulting kinetic energy is converted into electrical energy and is buffered, i.e., intermediately stored, if necessary, and then made available to the consumer upon a respective trigger. In this context, it is a preferred solution that the generator is attached to one or more rollers of the movable device to convert the kinetic energy into electrical energy. Alternatively, or additionally to the rollers, it is also possible, for example, to generate energy by opening and closing a door/the doors and/or a drawer. For this purpose, also supercapacitor and/or piezo elements can be used, for example.

The charge controller/rectifier must convert the induced AC voltage of the generator to a DC voltage. Further, it has to be assumed that the medical, preferably movable device, in particular the carriage/shelf cabinet, is not moved at a constant speed. For this reason, an appropriate charge control is required to appropriately protect the power accumulator when it is in the form of an energy storage unit, such as an accumulator, and to supply it with lower variations in the voltage.

In order to minimize the required energy of a consumer, preferably an electric consumer, an intelligent type of control of the consumers is additionally required. For example, it makes no sense that a transmitter intends to permanently send and/or receive. Since the consumers need hardly any energy in the standby mode, it is useful to activate them by defined trigger points only.

It is advantageous when the modular energy conversion system is provided and adapted so that the controller/charge controller and/or the generator itself is/are provided and configured so as to generate and/or convert the electrical energy as late as after a starting torque. In other words, it is advantageous when the generator starts generating power as late as during running. The rolling friction during running is substantially lower than the starting friction. This ensures that the drive moment of the dynamo does not additionally counteract during starting and accelerating.

Preferably, the energy conversion system is provided and adapted so that the controller is provided and configured to transmit the DC voltage to the energy storage unit only from a predefined voltage value. This offers the advantage of a more homogenous charging operation and, moreover, results in lower voltage variations to protect the energy storage unit.

Advantageously, the energy conversion system is provided and adapted so that the energy storage unit includes an accumulator or a capacitor, preferably a supercapacitor, the energy storage unit being especially provided and configured to make available capacities ranging from 50 mAH to 3000 mAH. This offers the advantage that, using this energy storage unit, sufficient energy, preferably when passing the sterile goods cycle, can be made available to the consumers.

It is preferred when the modular energy conversion system is provided and adapted so that the at least one trigger point is set by movement of the whole movable device and/or at least a part of the movable device. It is of advantage to set the trigger/trigger point only by movement, as this is accompanied by the energy conversion. Such movement is understood to be, for example, the opening or closing of a carriage/cabinet door or drawer and/or the starting of the movable device itself. Moreover, depending on the use case and a further type of consumer, it is preferred when further trigger points are set for the respective consumer, namely ideally such that there is no risk of the energy storage unit or the accumulator being fully discharged.

Preferably, the energy conversion system is provided and adapted so that the at least one electric component is provided and adapted for collection and/or transmission of data and/or information. Basically, it is the goal of the system to generate and to further transmit data about the content of the movable device, preferably of the shelf/cabinet carriage, as well as the position thereof in the sterile goods cycle. Accordingly, the consumer preferably consists of a system of antennas for data acquisition/retrieval and for data transfer. In particular, systems which require energy are installed.

Several exemplary configurations of the electric components and, resp., of the collection and/or transfer of data and/or information are antennas for reading out passive RFID tags, particularly NFC tags, which are capable of or adapted for reading out the carriage content and/or container content. Furthermore, wireless network technology modules such as modules using BLUETOOTH® Low Energy technology can be used for further communication with other models. The goal basically is to transmit the generated data into a cloud-based system. The transmission can be carried out via wireless technology such as BLUETOOTH® Low Energy brand technology using further firmly installed communication means, for example, which then can directly comprise a gateway or again can communicate with such gateway. The communication means may all be known devices facilitating communication. Another option is to directly send the data through 5G or LTE, instead of the gateway, alternatively also through WLAN, where appropriate. A gateway in information technology is understood to be a component—hardware and/or software—that establishes a connection between two systems. The term gateway implies that the transmitted data are processed.

It is of advantage when the collection and/or transfer of data and/or information takes place/is carried out at a predetermined interval and preferably while the medical, preferably movable, device is in motion. In other words, a trigger point for reading out the content in the movable device/carriage may be set after opening or closing the doors/drawers, for example, over a defined period of time, or when it is determined that the carriage rests after movement or, conversely, is moved again after rest. The readout time itself can be restricted to one second and can be repeated over a defined number in a defined interval, such as every 30 seconds.

The energy conversion system is preferred to be provided and adapted for the generator being in the form of a dynamo, further preferred a hub dynamo. As an alternative, all types of dynamos are imaginable. The different types of dynamos are adapted to convert the kinetic energy during movement of the medical movable device/the medical carriage/carriages from the rollers, for example, into electrical energy. The hub dynamo is especially preferred, because it is maintenance-free and has a high or optimum efficiency. Moreover, it generates the lowest friction losses. Alternative types of dynamos are tire-driven dynamos/rim dynamos, spoke dynamos and/or roller dynamos.

Advantageously, the energy conversion system is configured and arranged to communicate with at least one communicator in the environment so as to provide a tracking of the movable device. For tracking the movable device/the carriage itself, an infrastructure, preferably in the form of installed communicators, is facilitated in the AEMP (treatment unit of medical products)/ZSVA and, resp., in the hospital. These communicators communicate with the module when they are in appropriate local vicinity via wireless network technology such as BLUETOOTH® Low Energy brand technology, for example. Trigger points for activating these retrievals can, in turn, be the movement of the movable device/carriage itself, preferably the start-up or the rest (braking) of the movable device. Depending on the selected interval, preferably every 30 seconds, and the close network of the infrastructure, the accuracy is comparable to real-time tracking with an accuracy of less than one meter (<1 m).

It is generally assumed that, during a readout operation especially (a maximum of) between 1 and 5 watts are required. Depending on the set interval, the required capacity of the energy storage unit/accumulator is correspondingly low and can be appropriately provided.

Furthermore, the present disclosure relates to a (modular) energy conversion system in accordance with any one of the preceding aspects.

Furthermore, the present disclosure relates to a use of a (modular) energy conversion system for making available/providing a medical movable device including at least one electric component, preferably a cabinet carriage, a shelf carriage, a supply table and/or a removal carriage, the energy conversion system being disposed on preferably selected (manually) movable parts of the movable medical device in such a way that the kinetic energy generated during (manual) movement of the movable parts can be converted into electrical energy. The energy conversion system preferably comprises the following components:

a generator arranged and/or mounted on at least one component, preferably on at least one roller, of the movable medical device and is provided and configured to convert the kinetic energy occurring on this component into electrical energy,

a controller, preferably a charge controller, which is provided and configured to convert the electrical energy received as induced AC voltage into DC voltage, and

an energy storage unit which is provided and configured to intermediately store the electrical energy output by the controller as DC voltage and to transmit the electrical energy to a consumer in response to at least one trigger point. The above advantageous aspects are applicable also to this embodiment.

Moreover, the present disclosure relates to a monitoring system of a sterile goods cycle comprising:

a medical movable device including at least one electric component, in particular an electronic readout/collection and data transfer unit, in particular a sterile goods transport and/or storage device, preferably a cabinet carriage, a shelf carriage, a supply table and/or a removal carriage, in/on which sterile goods are stored with/as data carrier, the data carrier being adapted to be read out/collected by the electric component,

at least one computing unit/communicator to which the data from the electric component can be transmitted, and

an energy supply device for the electric component, wherein the energy supply device has a (modular) energy conversion system for converting kinetic energy into electrical energy comprising the following parts:

a generator which is arranged and/or mounted on at least one component, preferably on at least one roller of the movable device and is provided and configured to convert the kinetic energy (manually) generated on said component into electrical energy,

a controller, preferably a charge controller, which is provided and configured to convert the electrical energy received as induced AC voltage into DC voltage, and

an energy storage unit which is provided and configured to intermediately store the electrical energy output by the controller as DC voltage and to output the electrical energy to a consumer in response to at least one trigger point. The foregoing advantageous aspects are applicable also to this embodiment.

Basically, also other dynamos described in prior art can be used. Alternatively, also different generators are possible and can be used depending on the energy required. In addition to the generator, the supply of the energy can be provided via at least one photovoltaic module which correspondingly gains energy from the ambient light. The crucial fact is the self-sustaining supply of the shelf/transport carriages with energy to operate electronic components. Further, the energy conversion system can be arranged for energy generation/energy conversion also on other movable devices/device carriages or generally carriages in the medical field, such as hospital beds, surgical simulators such as simulators marketed under the trademark ENDOTOWER™, digital microscopes, ultrasonic apparatuses, respirators, IV poles and wheelchairs.

### BRIEF DESCRIPTION OF THE DRAWING FIGURES

FIG. 1 shows a view of an exemplary shelf carriage for use of the energy conversion system;

FIG. 2 shows a schematic view of the structure of the energy conversion system of the present disclosure;

FIG. 3 shows a schematic view of a sterile goods cycle as a basis for the assumed movement times of a movable device in particular according to the present disclosure.

The Figures are merely schematic and serve for the comprehension of the disclosure. Like elements are designated with like reference numerals.

### DETAILED DESCRIPTION

In the following, an embodiment of the present disclosure is described on the basis of the related Figures.

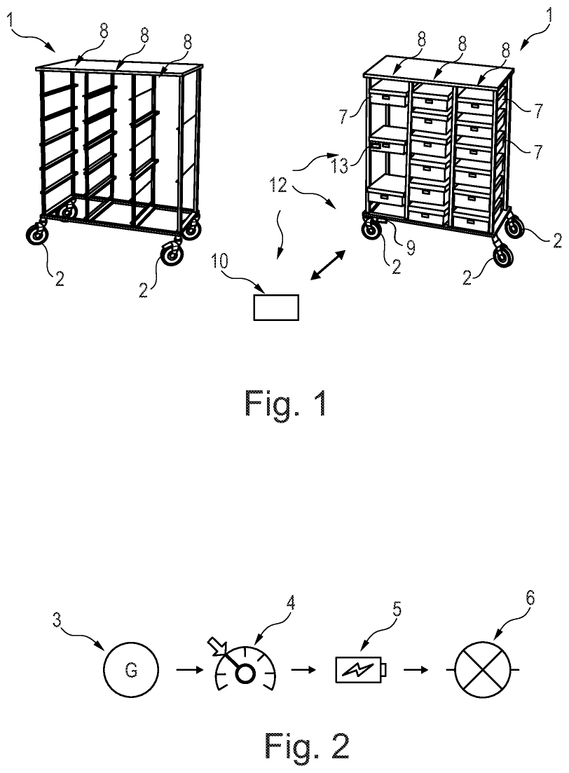

FIG. 1 shows a view of a shelf carriage for use of an energy conversion system, hereinafter referred to as medical device 1. The left picture of FIG. 1 shows an empty/non-filled medical, preferably movable device 1, and the right picture of FIG. 1 shows a medical device 1 loaded with sterile containers 7. This medical device 1 shown in FIG. 1 is equipped with four rollers, hereinafter referred to as parts 2 of the medical device 1, wherein at least two rollers/parts 2 are preferably provided with a parking brake. The medical device 1 is movable/pushable and exhibits a segment 8 for three sterile containers 7 up to a maximum height of 280 mm and two segments 8 for six sterile containers 7 up to a maximum height of 165 mm. The external dimensions in this case are 1030×630×1250 mm, but are merely exemplary. Basically, also soft packages can be included in the carriage, in particular when shelf carriages having a closed bottom are concerned.

FIG. 1 illustrates the complete module of the energy conversion system 9 being arranged/mounted on a part 2 of the medical, preferably movable, device 1. The generator 3 is provided to be in contact with the movable part 2 so as to convert the kinetic energy into electrical energy. The other components such as the controller 4, the energy storage unit 5 and the consumer 6 of the energy conversion system 9 can also be provided directly on the movable part 2 of the medical device 1 or can alternatively be provided elsewhere on the medical device 1. In both cases, an electric connection to the generator is provided. FIG. 1 further schematically illustrates an electric component 13 mounted on the medical device 1. Of preference, the electric component 13 is mounted (for collecting/transmitting the data) on the medical device 1. On the sterile containers 7/the soft packaging (content of the carriage) themselves a label, preferably RFID, which can be detected by the electric component 13, is arranged.

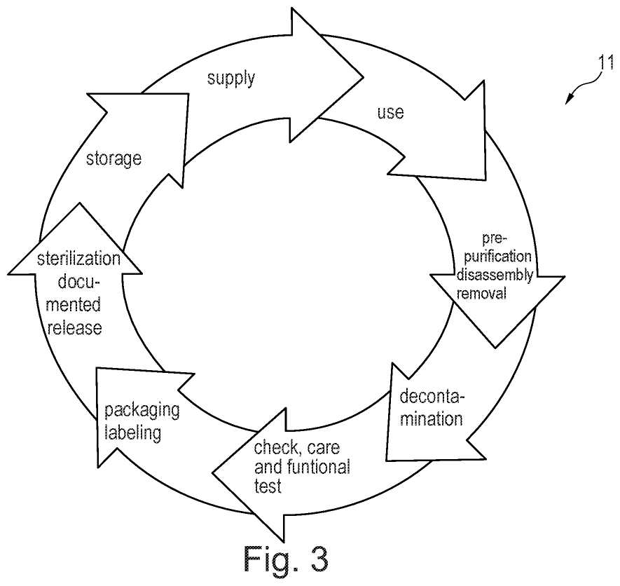

Furthermore, FIG. 1 shows a communicator 10 to which data and/or information collected by the consumer 6, preferably the electric consumer 6, are transmitted. Further, also plural communicators 10 are provided which are provided and adapted for tracking the medical, preferably movable device 1. In this way, it is possible to monitor the sterilization cycle (described in detail in FIG. 3). Hence, the monitoring system 12 includes at least one communicator 10, at least one electric component 13 and the energy conversion system 9.

FIG. 2 shows a schematic view of a structure of the preferably modular energy conversion system 9 of the present disclosure that is used, according to the disclosure, in a medical movable device 1. FIG. 2 illustrates a generator 3, a controller 4, an energy storage unit 5 and a consumer 6. The generator 3, which is mounted preferably as a hub dynamo on at least one part 2 in the form of a roller shown in FIG. 1 of the medical device 1, is provided and configured to convert the kinetic energy generated on the part 2 of the medical device 1 into electrical energy, wherein the kinetic energy in the exemplary case is generated by pushing/moving the medical device 1.

The kinetic energy converted into electrical energy by the generator 3 is converted, as induced AC voltage, into DC voltage by the controller/charge controller/rectifier 4 and is finally supplied to the energy storage unit 5. It is especially preferred when the controller 4 is provided and configured to appropriately control the voltage such that a voltage without, or at least with low, variations is supplied to the energy storage unit 5, for example by supply from a defined voltage only. The energy intermediately stored/buffered by means of the DC voltage is finally transmitted on demand, preferably depending on at least one trigger point to the consumer 6. The consumer or consumers 6 is/are electric components 13 provided in the medical device 1 which are provided and configured to read out the content of a sterile container 7 and, resp., the content of the whole medical device 1, for example, and to transmit this information.

In the present embodiment, it is provided that the energy conversion system 9 as shown in FIG. 2 has to be arranged on at least one of the movable components 2 of the medical device 1 according to FIG. 1 as a complete module comprising the generator 3, the controller 4 and the energy storage unit 5. Accordingly, the components of generator 3, controller 4 and energy storage unit 5 of FIG. 2 can be arranged/mounted as a module on at least one part 2 of the medical device 1, or the module can be arranged/mounted on the medical device 1, when it is already combined with a part 2 of the medical device 1. In other words, ideally the complete module is designed so that it is simply arranged to existing carriages by replacing the rollers or adapting them directly to the carriage bottom, for example (see also FIG. 1). In particular, the energy conversion system 9 is designed in the form of an exchangeable roller having a defined interface to the medical device 1 and being appropriately used.

FIG. 3 is a schematic view of a sterile goods cycle 11 as a basis for the assumed movement times of the medical device 1 according to the present disclosure. The sterile goods cycle in FIG. 3 includes the following working/process steps which are passed by the instruments/sterile goods in a medical device 1 and are adequately monitored by the monitoring system 12. The sterile goods cycle 11 includes the following eight steps:
supply,
use,
pre-purification/disassembly/removal,
decontamination,
check, care and functional test,
packaging/labeling,
sterilization/documented release, and
storage.

In this case, merely during the steps of storage and decontamination the medical device 1 is not moved so that in said two phases energy generation/conversion is not possible.

In the following, with respect to FIG. 3, assumptions concerning achievable amounts of energy are illustrated which are generated within a sterile goods treatment cycle 11. The movement of the medical device 1/carriage within the sterile goods cycle 11 forms the basis of generating energy.

The sterile goods cycle 11 is basically composed of the afore-mentioned eight individual steps. Accordingly, the essential movement of the medical device 1/carriage takes place between the storage and the decontamination. This means that during supply, use, pre-purification/disassembly/removal, check/care/functional test, packaging/labeling and sterilization/documented release the medical device 1 is in motion. The transport times, by way of example in two German hospitals, are known from the “Analysis of process and costs of alternative packaging options of sterile goods in hospitals—a case study in two German hospitals” by Krohn et al. These are:

transport from operating theater to ZSVA: 6:39 minutes

distance from ZSVA to airlock: 3:03 minutes

distance from airlock to operating theater: 3:58 minutes

Hence, it has to be assumed that the medical device 1 (the carriage) is pushed/is in motion over a complete sterile goods cycle 11 for at least 75% of this time. This corresponds to about 10 minutes.

By P=η*ω*M a possible capacity to be generated can be determined using an efficiency η of the generator 3, the angular speed w of the part 2 of the medical device 1 (the roller) and the torque M applied to the movable part 2 (the roller).

On the assumption that the carriage/medical device 1 is moved at 3 km/h, an angular speed of the part 2 of the medical device 1 of ω=1.76*2*η[1/s] is resulting with a roller diameter or, resp., diameter of the part 2 of the medical device 1 of 150 mm, which is common for a large shelf carriage. The torque is finally determined from the effective radius in the generator 3 and the resistance. The resistance is a question of interpretation. Therefore, in this context, an approximation is made on the basis of ergonomic issues.

According to DIN standard 33411, women can pull a maximum of 110 N and push a maximum of 140 N. Therefrom, according to DIN standard 33411, physical strength of the human being part 5 >Maximum static action forces<Table 11, it follows that the following practice-oriented forces are achieved during pulling:
95% of all men pull about 190 N,
50% pull about 275 N,
5% pull about 400 N, and
95% of all women pull about 110 N,
50% pull about 180 N,
5% pull about 260 N.

For pushing at a handle at a height of 1.35 m with safe stand, the following values are resulting:
95% of all men push about 310 N,
50% push about 510 N,
5% push about 770 N, and
95% of all women push about 140 N,
50% push about 250 N,
5% push about 400 N.

For damage-free and convenient handling of the loads, 15% of the maximum force should not be exceeded. Therefrom a minimum force of 16.5 N is resulting which can be easily applied. This force can be divided into the rolling resistance of the medical device 1/carriage (the starting torque is left out of consideration, as the generator 3 (dynamo) is to be connected only at a later point in time) and the force available for driving the generator 3. Here it is assumed that 8 N are available.

With an assumed efficient diameter of the generator 3 of r=50 mm and an efficiency of the generator 3 and control of η=0.5, the following capacity is resulting:

$$P = η * ω \times M = 0.5 * 1.75 * 2 * π * 1 / s * 8 N * 50 mm * 1 m / 1000 mm = 2.2 W$$

Based on the fact that the consumer is switched for one second each time, with one watt power consumption 1320 operations are resulting, with five watts power consumption 264 operations are resulting which are possible with this amount of energy.

Thus, the energy conversion system 9 is capable, based on the afore-made assumptions, of working completely self-sustaining and of supplying all electric components which are provided on/in the medical device 1 with power/energy.

Ideally, the complete energy conversion system 9/module is designed and configured so that it can be arranged on at least one medical device (carriages), for example by replacing the parts 2 (rollers) of the medical device 1 or adapting the same directly to the bottom of the medical device 1 (carriage bottom).

1. A medical transport and/or storage device comprising:
an energy conversion system for converting a kinetic energy into an electrical energy for supplying the electrical energy to at least one electric component of the medical transport and/or storage device; and
an energy storage unit configured to intermediately store the electrical energy and to transmit the electrical energy in response to at least one trigger point to a consumer,
the electrical energy being generated from movement of the medical transport and/or storage device or at least one part of the medical transport and/or storage device.

2. The medical transport and/or storage device according to claim 1, wherein the medical transport and/or storage device is a cabinet carriage, shelf carriage, supply table or removal carriage.

3. The medical transport and/or storage device according to claim 1, further comprising:
a generator arranged and/or mounted on at least one part or at least one roller of the medical storage and/or transport device, the generator configured to convert the kinetic energy into an electrical energy; and
a controller configured to convert the electrical energy into a DC voltage and output the electrical energy,
the energy storage unit configured to intermediately store the electrical energy output from the controller as DC voltage and output the electrical energy in response to said at least one trigger point to a consumer.

4. The medical transport and/or storage device according to claim 3, wherein the controller and/or the generator is/are provided and configured so as to generate and/or convert the electrical energy as late as after a starting torque.

5. The medical transport and/or storage device according to claim 3, wherein the controller is provided and configured to transmit the DC voltage to the energy storage unit only from a predefined voltage value onwards.

6. The medical transport and/or storage device according to claim 3, wherein the energy storage unit includes an accumulator and/or a capacitor.

7. The medical transport and/or storage device according to claim 1, wherein the energy conversion system is provided and adapted so that the at least one trigger point is set by movement of the medical transport and/or storage device and/or said at least of one part of the medical transport and/or storage device.

8. The medical transport and/or storage device according to claim 1, wherein the energy conversion system is provided and adapted so that the at least one electric component is provided and adapted for a collection and/or transmission of data and/or information.

9. The medical transport and/or storage device according to claim 8, wherein the energy conversion system is provided and adapted so that said collection and/or transmission of data and/or information is carried out at a predetermined interval. The medical transport and/or storage device according to claim 3, wherein the energy conversion system is provided and adapted so that the generator is in the form of a dynamo.

11. The medical transport and/or storage device according to claim 1, wherein the energy conversion system is configured and provided to communicate with at least one communicator so as to provide a tracking of the medical transport and/or storage device.

12. A monitoring system of a sterile goods cycle comprising:
a medical transport and/or storage device for sterile goods, the medical transport and/or storage device including at least one electric component or an electronic readout/collection and data transfer unit, the medical transport and/or storage device configured to store sterile goods with/as a data carrier adapted to be read out/collected using the electronic readout/collection and data transfer unit;
at least one computing unit/communicator to which data from the electronic readout/collection and data transfer unit is transmittable;
an energy supply device for the at least one electric component or the readout/collection and data transfer unit, which has an energy conversion system for converting a kinetic energy into an electrical energy;
a generator arranged or mounted on at least one movable part of the medical transport and/or storage device and being configured to convert a kinetic energy generated by the at least one movable part into an electrical energy,
a controller configured to convert the electrical energy into a DC voltage; and
an energy storage unit configured to intermediately store the electrical energy output by the controller or charge controller as DC voltage and to output the electrical energy in response to at least one trigger point to a consumer.

13. The monitoring system according to claim 12, wherein the transport and/or storage device is a cabinet carriage, a shelf carriage, a supply table or a removal carriage.

14. A method for supplying a medical transport and/or storage device with electrical power, the medical transport and/or storage device comprising at least one electronic component and an energy conversion system, the medical transport and/or storage device configured to transport and store sterile goods, the method comprising the steps of:
moving the medical transport and/or storage device or at least one part of the medical transport and/or storage device to generate a kinetic energy;
converting the kinetic energy to an electrical energy with the energy conversion system;
intermediately storing the electrical energy in an energy storage unit arranged in the medical storage and/or transport device; and
transmitting the electrical energy in response to at least one trigger point to a consumer.

15. The method according to claim 14, wherein the transport and/or storage device is a cabinet carriage, a shelf carriage, a supply table or a removal carriage.

16. A medical device for transporting and storing sterile goods, the medical device comprising:
at least one electric component or an electronic readout/collection and data transfer unit, wherein sterile goods are stored in or on the medical device with a data carrier adapted to be read out/collected using the at least one electric component or the electronic readout/collection and data transfer unit;
an energy supply device for the electric component or the electronic readout/collection and data transfer unit, the energy supply device comprising an energy conversion system, the energy conversion system comprising:
a generator arranged or mounted on at least one movable part of the medical device and configured to convert a kinetic energy generated by movement of the at least one movable part into an electrical energy;
a controller configured to convert the electrical energy into DC voltage; and
an energy storage unit configured to receive the electrical energy from the controller, intermediately store said electrical energy from the controller, and transmit said electrical energy from the controller in response to at least one trigger point to a consumer.

17. The medical device according to claim 16, wherein the medical device is a cabinet carriage, a shelf carriage, a supply table or a removal carriage.
